SEQUENCE LISTING (2 sequences)

SEQ ID NO: 1 - DNA, 525 bp, Leucosporidium golubevii
  source 1..525: mol_type = genomic DNA
gtgaatattagcgcatctcttcggagagcgtgacctccactttctaactctgtgcatttatttggcggctctgaagatgtaacagtctacttagctgcggctcattttataacactagttaaagtatgtaacgaaatatcgaaacaaaaaaaaactttcaacaacggatctcttggcttgctcatcgatgaagaacgcagcgaaatgtgataagtaatgtgaattgcagaattcagtgaatcatcgaatctttgaacgcaccttgcgctccgtggtattccgcggagcatgtctgtttgagtgtcatgaactcttcaacccaccagtttcttgtaaattggattggtgtttggattttgagtgttgctattcctagttgaatcagctcattcgtaatatattagcatctctaattcgaactcggattgactcagtgtaatagactattcgctgaggacacgctctttgtagtgtggccgaatgagatctcagtagaagcttccaactactttagtcaactttaga

SEQ ID NO: 2 - DNA, 524 bp, Leucosporidium golubevii
  source 1..524: mol_type = genomic DNA
gtgaatattagcgcatctcttcggagagcgtgacctccactttctaactctgtgcatttatttggcggctctgaagatgtaacagtctacttagctgcggctcattttataacactagttaaagtatgtaacgaaatatcgaaacaaaaaaaaactttcaacaacggatctcttggcttgctcatcgatgaagaacgcagcgaaatgtgataagtaatgtgaattgcagaattcagtgaatcatcgaatctttgaacgcaccttgcgctccgtggtattccgcggagcatgtctgtttgagtgtcatgaactcttcaacccaccagtttcttgtaaattggattggtgtttggattttgagtgttgctattcctagtgaatcagctcattcgtaatatattagcatctctaattcgaactcggattgactcagtgtaatagactattcgctgaggacacgctctttgtagtgtggccgaatgagatctcagtagaagcttccaactactttagtcaactttaga

(19) United States
(12) Patent Application Publication
(10) Pub. No.: US 2026/0218250 A1
(43) Pub. Date: Jul. 30, 2026

(54) NOVEL YEAST BELONGING TO GENUS LEUCOSPORIDIUM AND FAT PRODUCTION METHOD USING SAME

(71) Applicant: IDEMITSU KOSAN CO.,LTD., Tokyo (JP)

(72) Inventors: Shinzo MAYUZUMI, Tokyo (JP); Tomoko ISHII, Tokyo (JP); Shuichiro KIMURA, Tokyo (JP)

(73) Assignee: IDEMITSU KOSAN CO.,LTD., Tokyo (JP)

(21) Appl. No.: 19/141,718

(22) PCT Filed: Dec. 22, 2023
(86) PCT No.: PCT/JP2023/046267

§ 371 Date: Jun. 20, 2025

(30) Foreign Application Priority Data
Dec. 23, 2022 (JP) 2022-206911

(51) Int. Cl. C12P 7/64; C10L 1/18; C12N 1/165; C12R 1/645

(52) U.S. Cl. CPC C12P 7/64; C10L 1/1802; C12N 1/165; C12P 2203/00; C12R 2001/645

(57) ABSTRACT

A method for producing fats and oils. A yeast belonging to Leucosporidium golubevii, especially Leucosporidium golubevii IS-300 (NITE BP-03675) strain or its related strain is cultured in a medium containing sugar to produce fats and oils from the sugar, and the resulting fats and oils is collected. The medium may contain a saccharified product of lignocellulosic biomass.

### TECHNICAL FIELD

The present invention relates to a method of producing fats and oils using yeast and a novel yeast used in the method.

### BACKGROUND TECHNOLOGY

Vegetable oils and animal fats have been widely used as green fuel materials so far, but competition with food and the destruction of tropical rainforests due to palm plantations are becoming increasingly problematic. Therefore, the production of fats and oils from lignocellulosic biomass such as agricultural residues by microalgae or heterotrophic microorganisms (yeasts and molds) has been attracting attention. However, the high cost of both of these methods is an issue, and it is necessary to improve the production efficiency (oil-to-sugar yield) of fats and oils produced from raw materials by heterotrophic microorganisms in order to overcome this problem.

As described in Non-Patent Document 1, it has been reported that Leucosporidium creatinivorum, which belongs to the genus Leucosporidium, has the highest fats and oils production ability among many yeasts, but yeasts belonging to the genus Leucosporidium has the issue of relatively low growth temperature (approximately 20° C.).

### PRIOR ART DOCUMENTS

### Atent Documents

[atent document 1] Filippucci et al. Biotechnol Biofuels (2016) 9:259, DOI 10.1186/s13068-016-0672-1

### SUMMARY OF INVENTION

### Problem to be Solved by the Invention

As mentioned above, some strains of yeast in the genus Leucosporidium have been reported to be highly productive of fats and oils, but they are hypothermic, which poses a challenge for industrial use. Therefore, an object of the present invention is to provide a method for producing fats and oils using a mesophilic yeast that is advantageous in industrial production among yeasts of the genus Leucosporidium, and to provide a new yeast strain that can be used in said method.

### Means to Solve the Problem

In order to solve the above problem, the inventors have studied the yeast belonging to Leucosporidium golubevii and found that it is superior in the production of fats and oils. They also found that among the strains belonging to Leucosporidium golubevii, there were strains with relatively high temperature tolerance. In particular, a new oleaginous yeast IS-300 strain, was found to be more temperature tolerant than other Leucosporidium species and actively grew and produced fats and oils even under 28° C. culture conditions. Based on these findings, the present invention was completed.

The present invention provides a method for producing fats and oils, comprising culturing a yeast belonging to Leucosporidium golubevii in a medium containing sugar to produce fats and oils from the sugar, and collecting the resulting fats and oils.

Here, the medium containing sugar may be a medium containing saccharified product of lignocellulosic biomass.

The incubation temperature may be 10° C. to 30° C. or 25° C. to 30° C.

The sugar concentration in the medium may be 50 g/L to 500 g/L or 200 g/L to 500 g/L.

The yeast belonging to Leucosporidium golubevii may be Leucosporidium golubevii IS-300 (NITE BP-03675) strain or a related strain thereof whose sequence including the 5.8S rDNA region, ITS1 region, and ITS2 region is at least 95% identical to SEQ ID NO: 1, and whose fats and oils production ability at 28° C. is equivalent to the fats and oils production ability at 24° C. The invention also provides a new yeast strain, Leucosporidium golubevii IS-300 (NITE BP-03675), which is an excellent fats and oils producer.

The fats and oils obtained by the production method can be used as raw materials for biofuels, bionaphtha, food oil, lubricating oil, or surfactants.

The invention also relates to fats and oils produced by the aforementioned production method and biofuels or bionaphtha made from said fats and oils.

### Advantageous Effects of the Invention

Leucosporidium golubevii, especially Leucosporidium golubevii IS-300 strain, can be used to efficiently produce fats and oils from sugars such as biomass saccharified solution. In particular, since IS-300 strain is highly temperature tolerant, the fermentation production of fats and oils by culturing IS-300 or its related strains can be expected to suppress cooling energy during culture and increase the growth rate and fats and oils production rate by raising the culture temperature, thereby enabling efficient production of fats and oils.

The fats and oils produced by the method are not only used as GHG-reducing biofuel and bionaphtha raw materials, but can also be used as raw materials for food oils, lubricant base materials, and surfactants with low environmental impact.

### BRIEF DESCRIPTION OF THE DRAWINGS

FIG. 1 Microscopic image of IS-300 strain (photograph).

FIG. 2 Figure showing the phylogenetic position of the novel oleaginous yeast Leucosporidium golubevii IS-300 strain in the molecular phylogenetic tree of the genus Leucosporidium (5.8S rDNA and ITS sequences, NJ method). GenBank Accession numbers of DNA sequences used for phylogenetic analysis are shown in the figure. The number for each node is the bootstrap value of 1,000 replications.

FIG. 3 Figure showing the dry cell weight and triacylglyceride production of each strain of Leucosporidium spp. at 24° C. for each 24-hour period.

FIG. 4 Figure showing the dry cell weight and triacylglyceride production of each strain of Leucosporidium spp. at 28° C. every 24 hours.

FIG. 5 Figure showing the CYM medium-based nitrogen source species study, showing changes over time in dry cell weight in flask culture.

FIG. 6 Figure showing the CYM medium-based nitrogen source species study, showing changes over time in triacylglyceride production in flask culture.

FIG. 7 Figure showing the change over time of dry cell weight in flask culture under various conditions based on YSM medium with different nitrogen sources.

FIG. 8 Figure showing the change over time of triacylglyceride production in flask culture under various conditions based on YSM medium with different nitrogen sources.

FIG. 9 Figure showing the concentration of ammonium sulfate based on CYM medium and the change over time of the dry cell weight in flask culture.

FIG. 10 Figure showing the variation over time of triacylglyceride production in flask culture, evaluating the concentration of ammonium sulfate based on CYM medium.

FIG. 11 Figure showing the various carbon source studies based on CYM medium, showing changes over time in dry cell weight in 250 mL jar fermenter culture.

FIG. 12 Figure showing the various carbon source studies based on CYM medium, showing changes over time for each carbon source in 250 mL jar fermenter culture.

FIG. 13 Figure showing the changes over time in triacylglyceride production in various carbon source studies and 250 mL jar fermenter cultures based on CYM medium.

FIG. 14 Figure showing the changes over time in dry cell weight and triacylglyceride production in a 2 L jar fermenter culture using waste molasses.

FIG. 15 Figure showing the changes over time in sucrose, glucose, and fructose concentrations in a 2 L jar fermenter culture using waste molasses.

FIG. 16 Figure showing the changes over time in dried cell weight in a 250 mL jar fermenter culture under different incubation temperature conditions.

FIG. 17 Figure showing the changes over time in glucose concentration in 250 mL jar fermenter culture under various incubation temperature conditions.

FIG. 18 Figure showing the changes over time in triacylglyceride production in 250 mL jar fermenter culture under different culture temperature conditions.

FIG. 19 Figure showing the changes over time in dried cell weight in a 2 L jar fermenter culture under different culture pH conditions.

FIG. 20 Figure showing the changes over time in glucose concentration in 2 L jar fermenter cultures under various culture pH conditions.

FIG. 21 Figure showing the changes over time in triacylglyceride production in a 2 L jar fermenter culture under different culture pH conditions.

FIG. 22 Figure showing the initial glucose concentration study, showing changes over time in dry cell weight in 250 mL jar fermenter culture.

FIG. 23 Figure showing the initial glucose concentration study, showing changes over time in glucose concentration in 250 mL jar fermenter culture.

FIG. 24 Figure showing the initial glucose concentration study, showing changes over time in triacylglyceride production in 250 mL jar fermenter culture.

FIG. 25 Figure showing the initial glucose concentration study, showing a comparison of the yield of triacylglyceride per sugar at different glucose concentrations at the beginning of the incubation.

FIG. 26 Figure showing the initial glucose concentration study (high concentration), showing changes over time in dry cell weight in 250 mL jar fermenter culture.

FIG. 27 Figure showing the initial glucose concentration study (high concentration), showing changes over time in glucose concentration in 250 mL jar fermenter culture.

FIG. 28 Figure showing the initial glucose concentration study (high concentration), showing changes over time in triacylglyceride production in 250 mL jar fermenter culture.

FIG. 29 Figure showing the initial glucose concentration study (high concentration), showing a comparison of the triacylglyceride yield per sugar at different glucose concentrations at the beginning of incubation.

### EMBODIMENTS FOR CARRYING OUT THE INVENTION

The method for producing fats and oils comprises culturing yeast belonging to Leucosporidium golubevii in a medium containing sugar to produce fats and oils from the sugar and collecting the resulting fats and oils.

Fats and oils, also called acylglycerols, are produced by fermentation from sugars by yeasts belonging to Leucosporidium golubevii and are not restricted to any type of fats and oils, include triacylglycerols, diacylglycerols and monoacylglycerols.

Leucosporidium golubevii is not limited to any yeast taxonomically classified as Leucosporidium golubevii, but IS-300 strain (NITE BP-03675) is preferred.

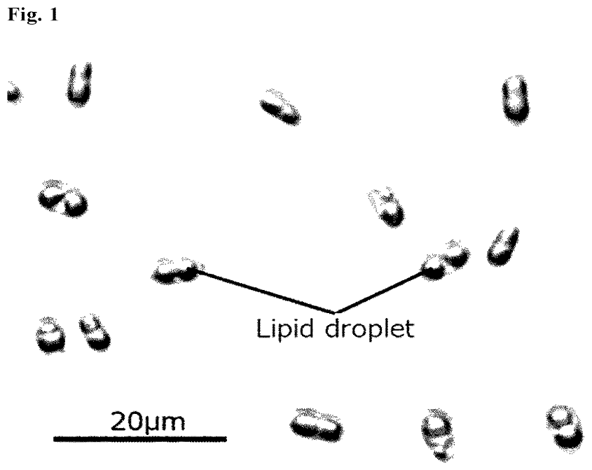

Leucosporidium golubevii IS-300 strain was isolated from the flower of Gnaphalium affine in Sodegaura, Chiba, Japan, and was deposited at NITE Patent Microorganisms Depository, National Institute of Technology and Evaluation (NITE) on Jun. 24, 2022. Microorganisms Depository (Room 122, 2-5-8 Kazusa-Kamashi, Kisarazu, Chiba, Japan) under the deposition number NITE BP-03675 in accordance with the Budapest Treaty. Colonies are white, shiny, smooth, and form a viscous substance on YPD agar medium. Cells are oval and form oil droplets (FIG. 1).

Among the DNA sequences encoding the rRNA of IS-300 strain, the 5.8S rDNA region and the ITS1 (internal transcribed spacer1) and ITS2 (internal transcribed spacer1) regions are shown in SEQ ID NO: 1.

In the method for producing fats and oils, a related strain of IS-300 strain can also be used. A related strain of IS-300 strain whose ability to produce fats and oils at a medium (mid) temperature (e.g., 28° C.) is equivalent to that at a low temperature (e.g., 24° C.) can also be used. Here, “the fats and oils production ability at 28° C. is equivalent to that at 24° C.” means that the fats and oils production ability of the related strain of IS-300 strain when cultured at 28° C. in a sugar-containing medium is at least 50%, preferably at least 80%, of that of the same strain when cultured at 24° C. in the same sugar-containing medium for the same period.

The term “related strain” means a strain in which the nucleotide sequence of the 5.8S rDNA region and the ITS1 and ITS2 regions is at least 95%, preferably 98%, and more preferably 99% identical to SEQ ID NO: 1.

Related strains can be naturally occurring strains or strains bred by mutation or other means from the IS-300 strain. Such related strains include Leucosporidium golubevii CBS 9652.

The medium is not limited as long as it contains sugar and is capable of growing Leucosporidium golubevii.

The sugars may be monosaccharides or polysaccharides. The medium may also contain sugar-containing raw materials. Various sugars can be used, including glucose, sucrose, fructose, xylose, mannose, soluble starch, glycerol, mannitol, and others. Examples of sugar-containing raw materials include molasses and saccharified lignocellulosic biomass. Here, as lignocellulosic biomass, grassy and woody biomasses such as bagasse, corn stover, wheat straw, rice straw, switchgrass, napier grass, Erianthus, bamboo grass, and silvergrass, as well as waste wood, sawdust, bark, and waste paper can be suitably used. Lignocellulosic biomass contains cellulose and hemicellulose (hereinafter referred to as celluloses), and can be used as a raw material containing sugar by breaking down cellulose into sugar such as glucose and xylose using saccharification enzymes according to conventional methods.

The concentration of sugar in the medium may be 50 g/L to 500 g/L, preferably 60 g/L to 420 g/L, more preferably 100 g/L to 420 g/L. When the culture medium contains sugar-containing materials, the sugar concentration converted from the concentration of the sugar-containing materials should be adjusted to be in the above range.

Since IS-300 strain can utilize high concentrations of sugar, the sugar concentration in the medium may be 200 g/L to 500 g/L or 200 g/L to 400 g/L when culturing IS-300 strain or its related strains. This allows the concentration of fats and oils produced to be increased, thereby improving the efficiency of fat and oil production.

Preferably, the medium further contains a nitrogen source. The nitrogen source is not limited, but may be, for example, yeast extract, malt extract, meat extract, peptone, casamino acid, corn steep liquor, etc. Nitrogen sources may be ammonium sulfate, urea, potassium nitrate, etc.

Other than these, inorganic salts such as magnesium salts (magnesium sulfate heptahydrate, etc.), calcium salts (calcium chloride, etc.), phosphates (potassium phosphate, etc.), iron salts (iron sulfate, etc.), copper salts (copper sulfate, etc.) and sodium salts (sodium chloride, etc.) are preferably included as components.

Generally, a high C/N ratio (ratio of nitrogen to carbon source in the medium, carbon source/nitrogen source) is desirable for high production of fats and oils, but yeasts belonging to the genus Leucosporidium are less affected by C/N ratio in fats and oils production, and the nitrogen concentration at which the yeast can grow is not particularly limited. For example, a C/N ratio (ratio of molar concentration of carbon to molar concentration of nitrogen) of 10 to 300 is preferred, 10 to 150 is more preferred, 10 to 100 is even more preferred, and 30 to 100 is especially preferred.

The culture temperature can be any temperature at which yeasts belonging to Leucosporidium golubevii can grow. For example, 10° C. to 30° C. is preferred, 20° C. to 30° C. is more preferred, and 20 to 28° C. is even more preferred. IS-300 strain can grow at medium (mid) temperature, the culture temperature may be 25° C. to 30° C. Culturing at 20° C. to 28° C. has the advantage of inhibiting the growth of contaminant bacteria and improving the culture efficiency.

The pH of the medium can be any pH at which strains belonging to Leucosporidium golubevii can grow, and is not limited. For example, pH 3.5 to 7.0 is preferred, pH 3.5 to 6.0 is more preferred, and pH 4.0 to 6.0 is even preferred.

The culture method of Leucosporidium golubevii is not limited, and the cells may be cultured by directly inoculating them into the culture medium, or the pre-culture solution obtained by pre-culturing may be inoculated into the liquid medium and cultured. Alternatively, cells that have been cultured on solid medium may be inoculated into the liquid medium and cultured. Known culture media for conventional yeast culture may be used, such as PDA and YPD media.

The method of culture is not restricted as long as the strain belonging to Leucosporidium golubevii can grow. For example, agitation culture, shaking culture, static culture, etc. can be used. The culture methods include, for example, batch culture, fed-batch culture, and continuous culture.

The incubation time of the strain belonging to Leucosporidium golubevii can be determined according to the amount of fats and oils desired, e.g., 1 day or longer, and 4 days or longer is preferred. In the case of a long incubation period, it is preferable to supplement sugar midway through the incubation. The upper limit of the incubation period is not limited, but can be up to 30 days, for example.

The oil fats and oils can be collected from the cells after culture by conventional known methods. For example, the cells may first be separated from the culture medium by centrifugation, filtration, or other means, and then the fats and oils may be extracted from the cells with an organic solvent such as normal hexane.

The obtained fats and oils may be subjected to a refining process. The refining process may be a collective refining of fats and oils or a refining of a single component of fats and oils. For example, industrial methods of refining fats and oils can be applied, such as adding water or acid to the fraction containing fats and oils obtained in the above collection process to remove precipitated gums, removing free fatty acids by adding alkali, and decolorizing by using activated clay. Silica gel chromatography may be repeated to further separate and refine the components.

The resulting fats and oils may be used as-is or refined for use as edible fats and oils, or as raw materials for biofuels, bionaphtha, lubricating oil, or surfactants. Biodiesel can be obtained by performing the method of the present invention using biomass such as edible sugar, molasses, or saccharified lignocellulosic biomass as a raw material, and performing transesterification of the resulting fats and oils with methanol (FAME as product), or biodiesel, bio-jet fuel, and bionaphtha can be obtained by hydrodeoxygenation of the obtained fats and oils, followed by isomerization and, if necessary, cracking (HVO and HEFA as products). The obtained fats and oils can be co-processed with petroleum raw materials in a petroleum refinery's hydrotreating unit to obtain diesel, jet fuel, kerosene, gasoline, etc., as a mixture with petroleum fuels.

Lubricating oil can be used, for example, as a lubricating oil base material or additive, and can be obtained by performing the method of the present invention and using the resulting fats and oils as it is, partially hydrogenating the unsaturated bond, or generating fatty acids to form alkanes.

Surfactants include, for example, anionic surfactants such as fatty acid metal salts, which can be obtained by performing the method of the present invention, producing fatty acids from the resulting fats and oils, and neutralizing them.

### EXAMPLES

The invention will be specifically described below with examples, but the invention is not limited to the following embodiments.

### Example 1

### <1> Acquisition of IS-300 Strain

The IS-300 strain was obtained from the flowers of Gnaphalium affine in Sodegaura, Chiba, Japan, using its ability to produce fats and oils from sugar as an index.

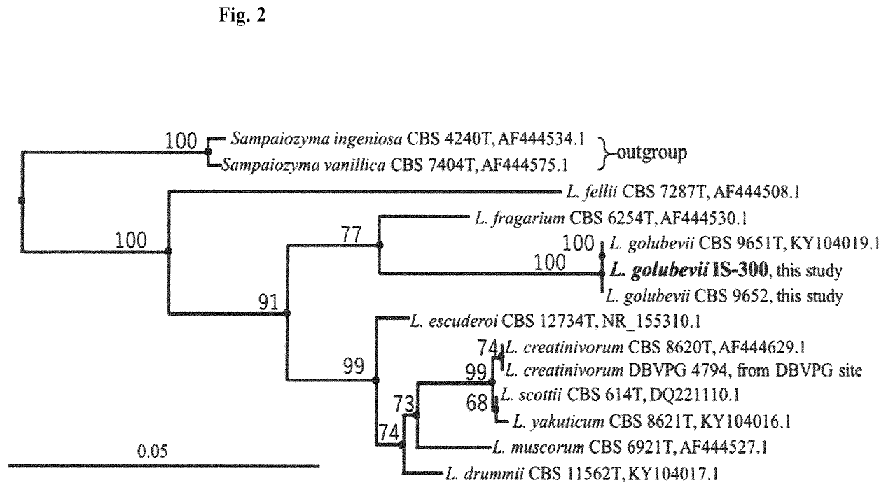

The DNA sequence encoding 5.8S rDNA region, ITS1 (internal transcribed spacer1) region, and ITS2 (internal transcribed spacer2) region of the rRNA of IS-300 strain was analyzed by DNA sequencing. The 5.8S rDNA region, ITS1 region, and ITS2 region of IS-300 strain were 100% identical to those of Leucosporidium golubevii type strain CBS 9651 and formed a monophyletic group with Leucosporidium golubevii CBS 9652 (FIG. 2). The IS-300 and CBS 9652 strains differed only by a single nucleotide. Therefore, IS-300 strain was identified as Leucosporidium golubevii. As described below, strain CBS 9651 differs from IS-300 strain in growth and fats and oils production ability at 28° C., so IS-300 strain is a different strain from CBS 9651, although the sequences of the 5.8S rDNA region and ITS1 and ITS2 regions are identical.

The nucleotide sequences of the 5.8S rDNA region and ITS1 and ITS2 regions of the new Leucosporidium golubevii IS-300 strain and Leucosporidium golubevii CBS 9652 are shown below.

>Leucosporidium golubevii IS-300
(SEQ ID NO: 1)
GTGAATATTAGCGCATCTCTTCGGAGAGCGTGACCTCCACTTTCT
AACTCTGTGCATTTATTTGGCGGCTCTGAAGATGTAACAGTCTAC
TTAGCTGCGGCTCATTTTATAACACTAGTTAAAGTATGTAACGAA
ATATCGAAACAAAAAAAAACTTTCAACAACGGATCTCTTGGCTTG
CTCATCGATGAAGAACGCAGCGAAATGTGATAAGTAATGTGAATT
GCAGAATTCAGTGAATCATCGAATCTTTGAACGCACCTTGCGCTC
CGTGGTATTCCGCGGAGCATGTCTGTTTGAGTGTCATGAACTCTT
CAACCCACCAGTTTCTTGTAAATTGGATTGGTGTTTGGATTTTGA
GTGTTGCTATTCCTAGTTGAATCAGCTCATTCGTAATATATTAGC
ATCTCTAATTCGAACTCGGATTGACTCAGTGTAATAGACTATTCG
CTGAGGACACGCTCTTTGTAGTGTGGCCGAATGAGATCTCAGTAG
AAGCTTCCAACTACTTTAGTCAACTTTAGA
>Leucosporidium golubevii CBS9652
(SEQ ID NO: 2)
GTGAATATTAGCGCATCTCTTCGGAGAGCGTGACCTCCACTTTCT
AACTCTGTGCATTTATTTGGCGGCTCTGAAGATGTAACAGTCTAC
TTAGCTGCGGCTCATTTTATAACACTAGTTAAAGTATGTAACGAA
ATATCGAAACAAAAAAAAACTTTCAACAACGGATCTCTTGGCTTG
CTCATCGATGAAGAACGCAGCGAAATGTGATAAGTAATGTGAATT
GCAGAATTCAGTGAATCATCGAATCTTTGAACGCACCTTGCGCTC
CGTGGTATTCCGCGGAGCATGTCTGTTTGAGTGTCATGAACTCTT
CAACCCACCAGTTTCTTGTAAATTGGATTGGTGTTTGGATTTTGA
GTGTTGCTATTCCTAGTGAATCAGCTCATTCGTAATATATTAGCA
TCTCTAATTCGAACTCGGATTGACTCAGTGTAATAGACTATTCGC
TGAGGACACGCTCTTTGTAGTGTGGCCGAATGAGATCTCAGTAGA
AGCTTCCAACTACTTTAGTCAACTTTAGA
Example 2

<2>Comparison with Other Leucosporidium Species

Each yeast strain grown on PDA (potato dextrose agar) was inoculated into YPD medium dispensed in 14 ml PS tubes and incubated for 48 hours at 24° C. and 200 rpm with shaking (pre-culture). The pre-culture solution was inoculated into 100 ml of YPD modified medium in a 500 ml baffled flask to a final concentration of OD₆₀₀=0.1 and incubated at 160 rpm (main culture). At this time, the incubation temperatures were set at 24° C. and 28° C., and antifoam agent (Antifoam 204, Sigma) was added as needed. One ml was sampled every 24 hours from the start of the main culture and used for dry cell weight and triacylglyceride determination.

### The Composition of Each Culture Medium is as Follows

PDA medium
Potato Dextrose broth (Difco) 24 g
Agar (Wako) 20 g
Distilled water 1 L
   YPD medium
Bacto peptone (Difco) 20 g
Yeast extract (Difco) 10 g
Glucose (Wako) 20 g
Distilled water 1 L
   YPD modified medium
Bacto peptone (Difco) 10 g
Yeast extract (Difco) 10 g
Glucose (Wako) 60 g
Distilled water 1 L

The samples were centrifuged (700 g, 5 minutes) and the resulting pellet was dried overnight in a freeze dryer. The dry cell weight was measured before using it for triacylglyceride production measurement. Triacylglyceride content was measured using the following method. To inactivate lipase in the dried cells, 260 μL of distilled water and 40 μL of 10× D-PBS (Wako) were added to resuspend the cells, which were then boiled for 10 minutes. Afterwards, 100 μL of 25 mg/mL Zymolyase solution (Nacalai Tesque, Zymolyase-20T) was added, and the mixture was incubated with shaking at 35° C. for 1 hour. Then, 200 μL of glass beads (Merck, G8772-500G) and 500 μL of hexane were added, and the mixture was shaken (TAITEC, E-36, at maximum speed) for 1 hour. After centrifugation (10,000 g, 10 minutes), the hexane layer was transferred to a new microtube, and an additional 500 μL of hexane was added and shaken for another hour. The hexane layer obtained after a second centrifugation (10,000 g, 10 minutes) was added to the microtube. The triacylglyceride content in the hexane extract was measured using an enzymatic assay (LabAssay Triglyceride, Wako, 290-63701).

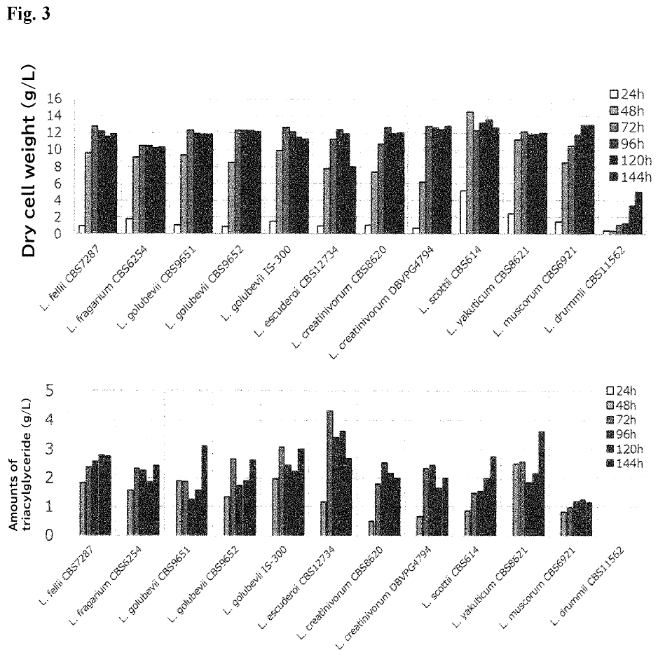

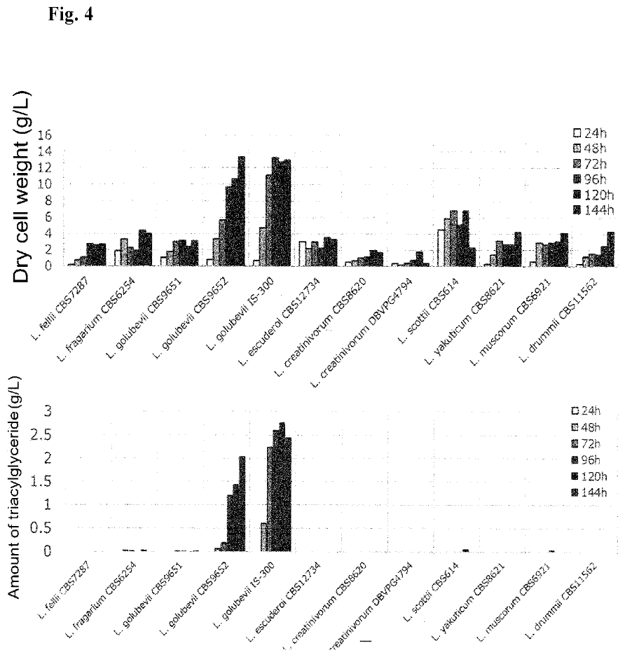

FIGS. 3 and 4 show the dry cell weight and triacylglyceride production of each strain every 24 hours.

At 24° C., all strains except L. drummii CBS 11562 showed vigorous growth and high triacylglyceride production (FIG. 3). On the other hand, at 28° C., all strains except Leucosporidium golubevii CBS9652 and IS-300 were significantly suppressed in growth, while IS-300 and CBS962 were only slightly suppressed in growth, and produced high levels of triacylglyceride (FIG. 4).

### Example 3

### Evaluation of Added Nitrogen Sources Based on CYM Medium for IS-300 Strain

### [Test Tube Culture (Pre-Culture)]

3 mL each of modified YPD113 medium (10 g/L Bacto Yeast Extract, 10 g/L Bacto Tryptone, 30 g/L Glucose) in 15 mL polystyrene (PS) tubes (@17 mm×100 mm) from AS ONE Corporation were inoculated with a small amount of IS-300 strain oleaginous yeast scraped from a YPD agar plate using a disposable loop. The rotary shaker NR-2 from TAITEC Corporation set at approximately 200 rpm was placed inside the SANYO INCUBATOR MIR-253, and shake culture was performed at 28° C. Pre-culture was carried out for 2 days.

[Evaluation of Nitrogen Sources Based on CYM Medium in a Triangular Flask Culture with 500 mL Baffle (Main Culture)]

CYM medium and modified CYM media in which ammonium sulfate in the CYM composition was replaced with either urea or potassium nitrate were prepared (Table 1). The amounts of ammonium sulfate, urea, or potassium nitrate added were adjusted to provide the same nitrogen concentration. During medium preparation, each medium was adjusted to pH 5.5 with 6N KOH, then brought to the required volume, and sterilized by filtration through a 0.22 μm pore size MF filter. Subsequently, 100 mL of each medium was aseptically dispensed into sterile 500 mL baffled Erlenmeyer flasks. The turbidity of the pre-culture in test tubes (OD600 nm value) was measured using a spectrophotometer, and an amount was inoculated to achieve an OD600 nm value of 0.05 at the start of the main culture. Shake culture was then performed at 28° C. and 180 rpm.

### [Table 1]

TABLE 1
CYM medium-based culture medium for
evaluating added nitrogen sources

| | Concentrations in each evaluation condition | | | | | |
|---|---|---|---|---|---|---|
| | | | | | CYM medium | |
| | | | CYM: medium | | Potassium | |
| | | | Urea | | nitrate | |
| Component | CYM medium | | condition | | condition | |
| Bacto Yeast Extract | 0.75 | g/L | 0.75 | g/L | 0.75 | g/L |
| (NH₄)₂SO₄ | 0.1 | g/L | — | | — | |
| CO(NH₂)₂ | — | | 0.045 | g/L | — | |
| KNO₃ | — | | — | | 0.15 | g/L |
| KH₂PO₄ | 0.4 | g/L | 0.4 | g/L | 0.4 | g/L |
| MgSO₄•7H₂O | 1.5 | g/L | 1.5 | g/L | 1.5 | g/L |
| CaCl₂•2H₂O | 0.22 | g/L | 0.22 | g/L | 0.22 | g/L |
| ZnSO₄ | 0.0003 | mg/L | 0.0003 | mg/L | 0.0003 | mg/L |
| MnSO₄•5H₂O | 0.029 | mg/L | 0.029 | mg/L | 0.029 | mg/L |
| CuSO₄•5H₂O | 0.025 | mg/L | 0.025 | mg/L | 0.025 | mg/L |
| MES (pH buffer) | 21.3 | g/L | 21.3 | g/L | 21.3 | g/L |
| Glucose | 60 | g/L | 60 | g/L | 60 | g/L |

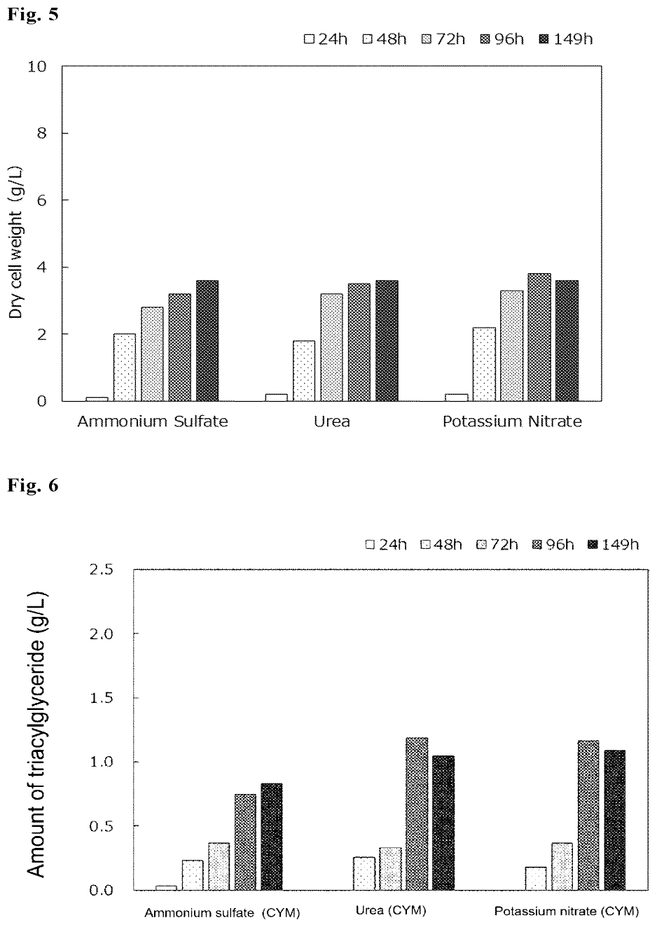

FIG. 5 shows the dry cell weight of IS-300 strain and FIG. 6 shows the triacylglyceride production over time. Cell growth and triacylglyceride production were confirmed under each condition using ammonium sulfate, urea, and potassium nitrate as the added nitrogen source, respectively.

### Example 4

Evaluation of added nitrogen sources based on YSM medium for IS-300 strain The pre-culture was carried out under the same conditions as in Example 3. For the main culture, YSM medium, and modified YSM medium in which the ammonium sulfate in the YSM medium composition was replaced with other nitrogen sources such as urea and potassium nitrate without changing the nitrogen (N) content in the medium were prepared (Table 2). Each medium was prepared in the same manner as in Example 3, and shake flask culture was carried out under the same culture conditions using baffled Erlenmeyer flasks.

TABLE 2
YSM medium based culture medium for
evaluating added nitrogen sources

| | Concentrations in each evaluation condition | | | | | |
|---|---|---|---|---|---|---|
| | YSM medium | | | | YSM medium | |
| | Ammonium | | YSM medium | | Potassium | |
| | sulfate | | Urea | | nitrate | |
| Component | condition | | condition | | condition | |
| Bacto Yeast Extract | 0.5 | g/L | 0.5 | g/L | 0.5 | g/L |
| (NH₄)₂SO₄ | 0.5 | g/L | 0.5 | g/L | 0.5 | g/L |
| CO(NH₂)₂ | — | | 0.23 | g/L | — | |
| KNO₃ | — | | — | | 0.77 | g/L |
| KH₂PO₄ | 0.092 | g/L | 0.092 | g/L | 0.092 | g/L |
| MgSO₄•7H₂O | 0.7 | g/L | 0.7 | g/L | 0.7 | g/L |
| Na₂HPO₄•2H₂O | 1.6 | g/L | 1.6 | g/L | 1.6 | g/L |
| CaCl₂•2H₂O | 0.066 | g/L | 0.066 | g/L | 0.066 | g/L |
| ZnSO₄ | 0.23 | mg/L | 0.23 | mg/L | 0.23 | mg/L |
| MnSO₄•5H₂O | 0.57 | mg/L | 0.57 | mg/L | 0.57 | mg/L |
| H₃BO₃ | 0.50 | mg/L | 0.50 | mg/L | 0.50 | mg/L |
| FeCl₃•6H₂O | 6.00 | mg/L | 6.00 | mg/L | 6.00 | mg/L |
| MES | 21.3 | g/L | 21.3 | g/L | 21.3 | g/L |
| Glucose | 60 | g/L | 60 | g/L | 60 | g/L |

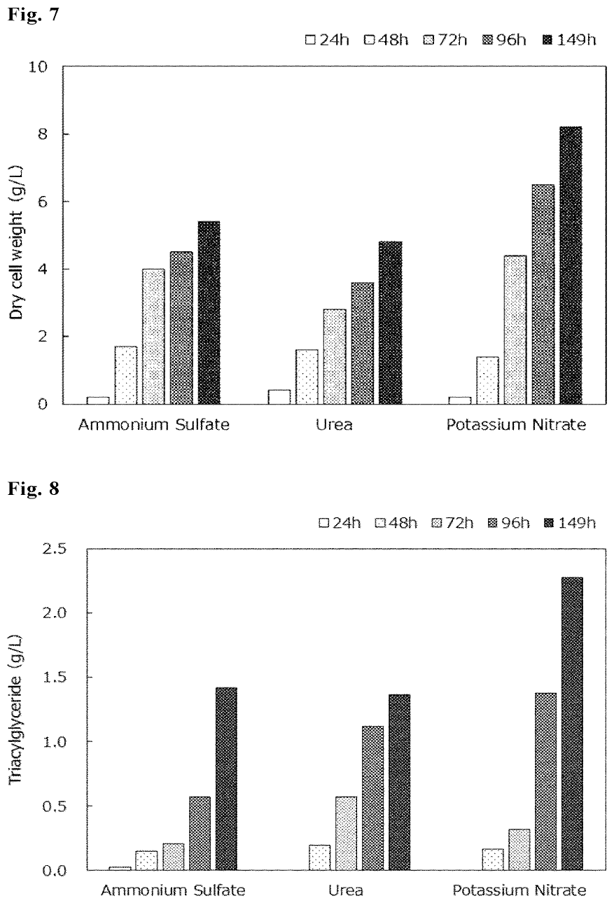

FIG. 7 shows the dry cell weight of IS-300 strain and FIG. 8 shows the change over time in triacylglyceride production. Cell growth and triacylglyceride production were confirmed under each condition using ammonium sulfate, urea, and potassium nitrate as the added nitrogen source, respectively.

### Example 5

### [Evaluation of Ammonium Sulfate Concentration Based on CYM Medium in a 500 mL Baffled Triangular Flask Culture (Main Culture)]

Pre-culture was carried out under the same conditions as Example 3. In the main culture, CYM medium (ammonium sulfate concentration 0.1 g/L, nitrogen concentration 0.103 g/L, C/N ratio=273) and CYM media with 10 times the ammonium sulfate concentration (ammonium sulfate concentration 1.0 g/L, nitrogen concentration 0.294 g/L, C/N ratio=96) and 100 times the ammonium sulfate concentration (ammonium sulfate concentration 10 g/L, nitrogen concentration 2.202 g/L, C/N ratio=13) were prepared (Table 3). The C/N ratio here indicates the ratio of molar concentration of carbon to molar concentration of nitrogen. Except for the medium composition, the media were prepared similarly to Example 3, and the same culture conditions were used to conduct shaking culture in baffled Erlenmeyer flasks.

TABLE 3
CYM medium-based culture medium for evaluating
ammonium sulfate concentration

| | Concentration in each evaluation condition | | | | | |
|---|---|---|---|---|---|---|
| | CYM medium | | CYM medium | | CYM medium | |
| | 0.1 g/L ammonium | | 1.0 g/L ammonium | | 10 g/L ammonium | |
| Component | sulfate condition | | sulfate condition | | sulfate condition | |
| Bacto Yeast Extract* | 0.75 | g/L | 0.75 | g/L | 0.75 | g/L |
| (NH₄)₂SO₄ | 0.1 | g/L | 1.0 | g/L | 10 | g/L |
| KH₂PO₄ | 0.4 | g/L | 0.4 | g/L | 0.4 | g/L |
| MgSO₄•7H₂O | 1.5 | g/L | 1.5 | g/L | 1.5 | g/L |
| CaCl₂•2H₂O | 0.22 | g/L | 0.22 | g/L | 0.22 | g/L |
| ZnSO₄ | 0.0003 | mg/L | 0.0003 | mg/L | 0.0003 | mg/L |
| MnSO₄•5H₂O | 0.029 | mg/L | 0.029 | mg/L | 0.029 | mg/L |
| CuSO₄•5H₂O | 0.025 | mg/L | 0.025 | mg/L | 0.025 | mg/L |
| MES (pH buffer) | 63.9 | g/L | 63.9 | g/L | 63.9 | g/L |
| Glucose | 60 | g/L | 60 | g/L | 60 | g/L |

*Total Nitrogen 10.9% BD Bionutrients-technical-manual

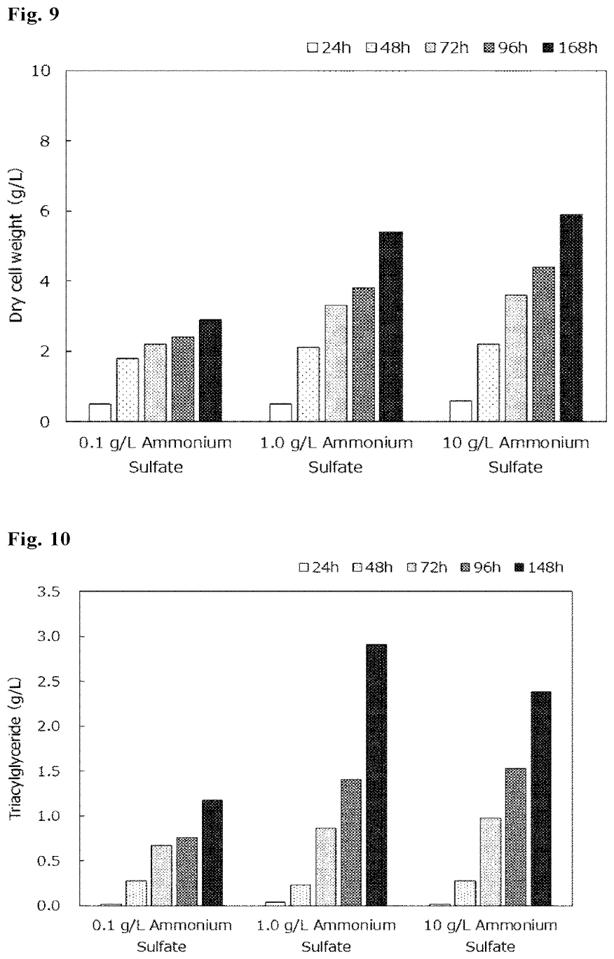

FIG. 9 shows the dry cell weight of IS-300 strain under the medium conditions of each ammonium sulfate concentration, and FIG. 10 shows the change over time in fats and oils production. Under the condition of 10 times higher ammonium sulfate concentration (ammonium sulfate concentration of 1.0 g/L, nitrogen concentration of 0.294 g/L, C/N ratio=96), the oil production increased more than twofold compared to the basic condition (ammonium sulfate concentration of 0.1 g/L, nitrogen concentration of 0.103 g/L, C/N ratio=273).

### Example 6

Confirmation of triacylglyceride production from each carbon source in a jar fermentor culture of IS-300 strain

### [Test Tube Culture (Pre-Culture)]

Each oleaginous yeast cell colony grown on YPD agar plates was lightly scraped with the tip of a disposable loop and inoculated into each 3.5 mL of YEL medium (5 g/L Bacto Yeast Extract, 30 g/L Glucose) contained in Asahi Glass Co., Ltd. 15 mL polystyrene (PS) tubes (@17 mm×100 mm). Culture was then carried out by shaking at 140 rpm at 28° C. for 2 days using the Iwashiya bio science Co., Ltd. multi-vessel shaker incubator MLU-4-GR-16.

[Comparison of Triacylglyceride Productivity from Each Carbon Source in 250 mL Jar Fermenter Culture (Main Culture)]

The jar fermenter used was an ABLE-Biott Co., Ltd. 250 mL 8-vessel bioreactor Bio Jr.8, with compressed air as the aeration source. Each main culture vessel was prepared with 95 mL of CYM-modified medium using glucose, xylose, arabinose, or glycerol as the carbon source (Table 4, autoclaved). The carbon source concentration was set to approximately 60 g/L (post-inoculation). After mixing the test tube culture and inoculating 5 mL of pre-culture to the main culture vessel, batch culture at 22° C. was initiated. The agitation speed was set to a minimum of 300 rpm, and the DO level was maintained at a minimum of 2 ppm through automatic control (DO cascade control). The aeration condition was set to 100 mL/min (1 vvm). From the start of culture, pH was automatically controlled to not fall below 5.0 using 0.5 N KOH, and if the pH rose, it was manually adjusted to the set pH using an external peristaltic pump with 0.1 N H₂SO₄. The main culture continued for 168 to 216 hours.

TABLE 4
modified CYM medium-based culture medium for evaluating
the carbon sources (after inoculation of pre-cultue)

| Component | Concentration | |
|---|---|---|
| CSL (Solulys 095E) * | 2.12 | g/L |
| (NH₄)₂SO₄ | 1.0 | g/L |
| KH₂PO₄ | 0.4 | g/L |
| MgSO₄•7H₂O | 1.5 | g/L |
| CaCl₂•2H₂O | 0.22 | g/L |
| ZnSO₄ | 0.0003 | mg/L |
| MnSO₄•5H₂O | 0.029 | mg/L |
| CuSO₄•5H₂O | 0.025 | mg/L |
| each carbon source | 60 | g/L |

* CSL: Corn Steep Liquor

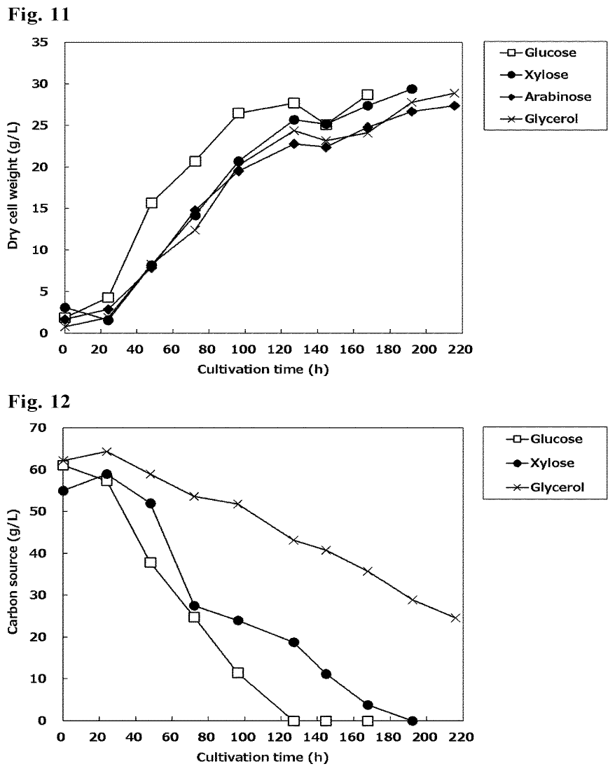

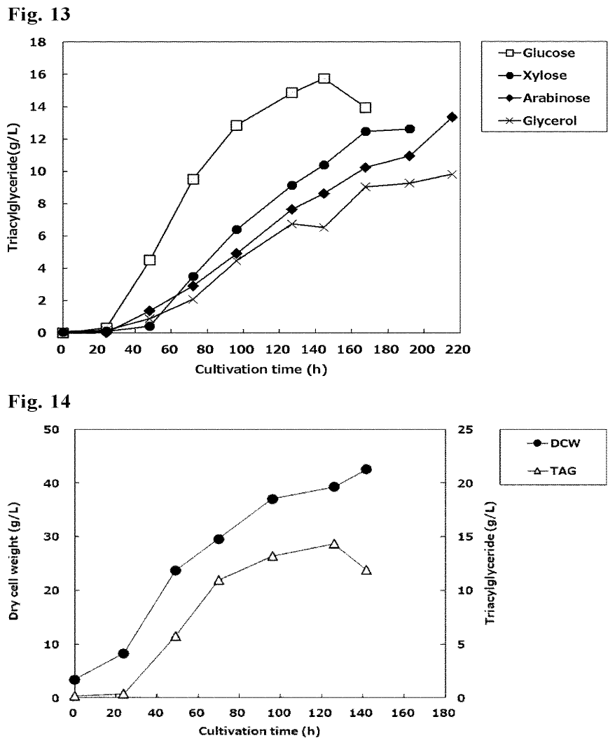

FIGS. 11, 12, and 13 show changes over time in the dry cell weight, carbon source concentration, and triacylglyceride production of IS-300 strain under each carbon source condition (only the arabinose concentration was not measured). The cell growth and triacylglyceride production were confirmed in all cultures that used C6 sugar glucose, C5 sugars xylose and arabinose, and glycerol as carbon sources. The condition using C6 sugar glucose as the carbon source showed the fastest microbial growth and triacylglyceride production, as well as the highest triacylglyceride yield.

### Example 7

Confirmation of Triacylglyceride Production from Waste Molasses in a Jar Fermenter Culture of IS-300 Strain

### [Test Tube Culture (Seed Culture)]

3.5 mL of YEL medium (5 g/L Bacto Yeast Extract, 30 g/L Glucose) in 15 mL polystyrene (PS) tubes (§ 17 mm×100 mm) from AS ONE Corporation were inoculated with a small amount of IS-300 strain oleaginous yeast scraped from a YPD agar plate using a disposable loop. The rotary shaker NR-2 from TAITEC Corporation set at approximately 200 rpm was placed inside the SANYO INCUBATOR MIR-253, and shake culture was performed at 28° C. Pre-culture was carried out for 2 days.

### [Flask Culture (Pre-Culture)]

After mixing the test tube cultures, 5 mL of seed culture was inoculated into 100 mL of YEL medium (5 g/L Bacto Yeast Extract, 30 g/L Glucose) in baffled 500 mL Erlenmeyer flasks, and culture was carried out at 28° C. for 2 days using the bio science Co., Ltd. multi-vessel shaker incubator MLU-4-GR-16 set at 180 rpm.

[Confirmation of Triacylglyceride Production from Waste Molasses in 2 L Jar Fermenter Culture (Main Culture)]

The jar fermenter used was a 2 L bioreactor Bioneer-Neo 2 L from B. E. MARUBISHI CO., LTD., and the main culture vessel was prepared with 950 mL of CYM-modified medium using waste molasses as the sugar source. The CYM-modified medium had the same composition as in Example 6. The sugar source concentration was set to approximately 60 g/L (total value of glucose, fructose, and sucrose). After inoculating the main culture vessel with 50 mL of pre-culture, batch culture at 22° C. was initiated. The agitation speed was set to a minimum of 300 rpm, and the DO level was maintained at a minimum of 24% (approximately 2 ppm) through automatic control (DO cascade control). The aeration condition was set to 1 L/min (1 vvm). From the start of culture, the pH was automatically controlled to 5.0 using 1.0 N KOH and 0.1 N H₂SO₄.

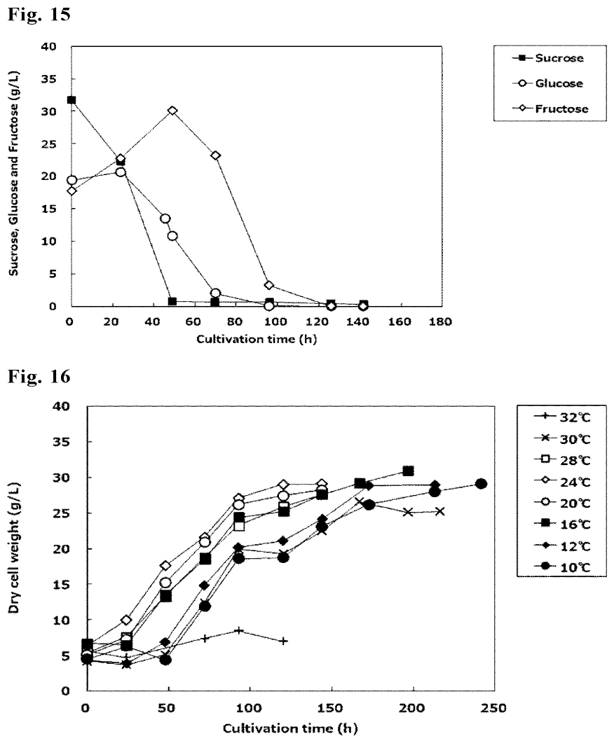

FIGS. 14 and 15 show the changes over time in dry cell weight, each sugar (sucrose, glucose, and fructose), and triacylglyceride production of IS-300 strain under conditions in which waste molasses was used as the sugar source, respectively. Cell growth and triacylglyceride production were also observed under conditions in which waste molasses was used as the carbon source. The sugar concentrations showed a decrease in sucrose concentration after the start of incubation, followed by a decrease in glucose concentration, and finally fructose concentration.

### Example 8

### Evaluation of Culture Temperature in Jar Fermenter Culture

### [Test Tube Culture (Pre-Culture)]

Pre-culture was performed in test tubes under the same conditions as in Example 6 as a seed culture.

### [Evaluation of Incubation Temperature in 250 mL Jar Fermenter Culture (Main Culture)]

The jar fermenter and aeration conditions used were the same as in Example 6. Each main culture vessel was prepared with 95 mL of modified CYM medium using reagent-grade glucose as the sugar source. The modified CYM medium had the same composition as in Example 6. In this case, the sugar concentration was set to approximately 60 g/L (post-inoculation). The culture temperature conditions were set to 10, 12, 16, 20, 24, 28, 30, and 32° C. After mixing the test tube cultures, 5 mL of pre-culture was inoculated into each main culture vessel, and batch culture was started under the various temperature conditions. The conditions for DO and agitation speed were the same as in Example 6. The main culture was continued for up to 144 hours.

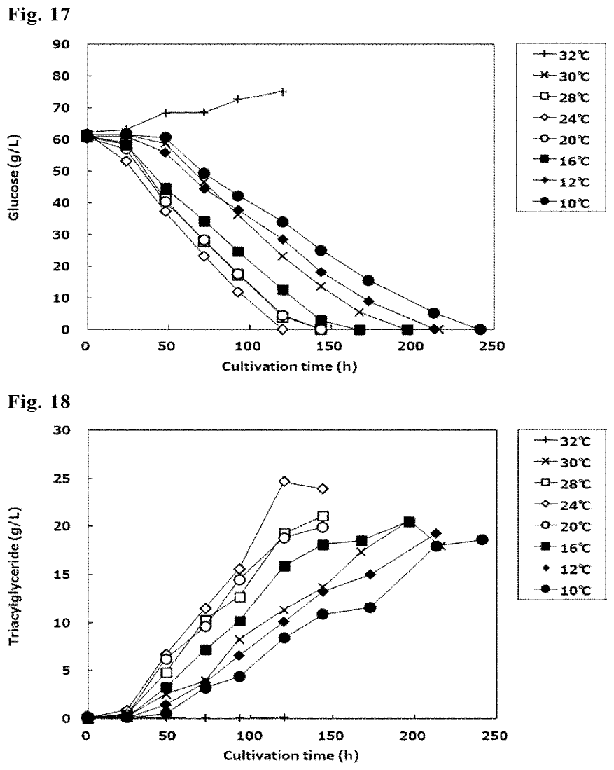

FIGS. 16, 17, and 18 show the changes over time in dry cell weight, glucose, and triacylglyceride production of IS-300 strain at various temperature conditions, respectively. Cell growth and triacylglyceride production were observed in the range of incubation temperatures from 10° C. to 30° C., with good results for triacylglyceride production rate and triacylglyceride production amount in the range of 20° C. to 28° C. No triacylglyceride production was observed at an incubation temperature of 32° C.

### Example 9

### Evaluation of Culture pH in Jar Fermenter Culture

### [Test Tube Culture (Seed Culture), Flask Culture (Pre-Culture)].

Seed culture and pre-culture were performed in test tubes and flasks under the same conditions as in Example 7.

### [Culture in 2 L Jar Fermenter Culture (Main Culture)]

The jar fermenter and aeration conditions used were the same as in Example 7. The main culture vessel was filled with 950 mL of CYM-modified medium using reagent-grade glucose as the sugar source. The CYM-modified medium had the same composition as in Example 6. The sugar concentration was set to approximately 60 g/L (post-inoculation). The culture pH conditions were set to pH 3.5, 4.0, 4.5, 5.0, 5.5, and 6.0. After inoculating the main culture vessel with 50 mL of pre-culture, batch culture at 22° C. was initiated. The agitation speed was set to a minimum of 300 rpm, and the DO level was maintained at a minimum of 24% (approximately 2 ppm) through automatic control (DO cascade control). The aeration condition was set to 1 L/min (1 vvm). From the start of culture, the pH was automatically controlled to each specified pH condition using 1.0 N KOH and 0.2 N H₂SO₄.

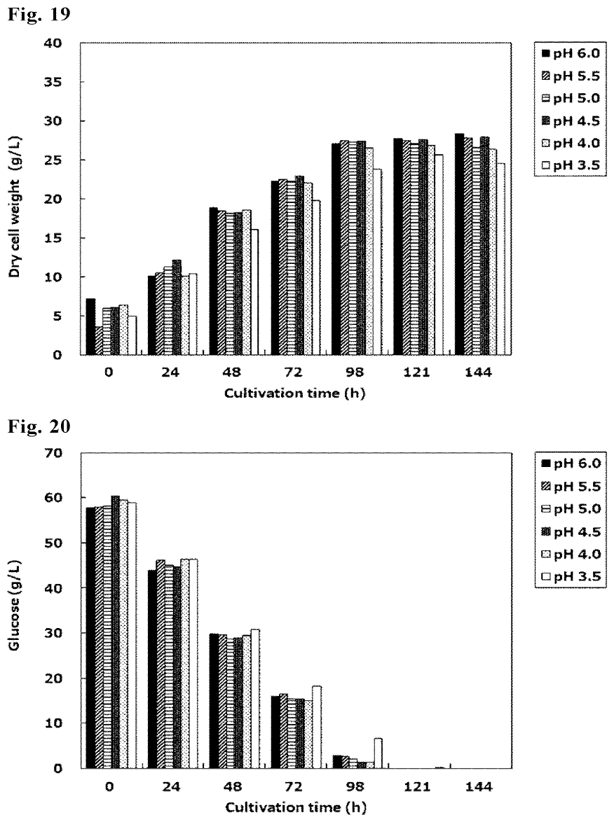

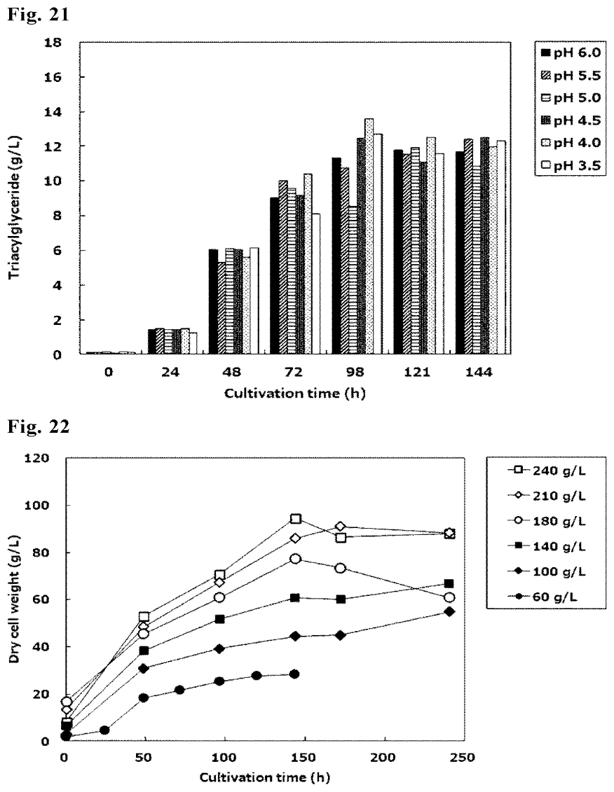

FIGS. 19, 20, and 21 show the changes over time in dry cell weight, glucose concentration, and triacylglyceride production of IS-300 strain at each culture pH condition, respectively. Cell growth and triacylglyceride production were observed at all culture pH conditions. At culture pH 3.5, glucose consumption tended to be slightly slower.

### Example 10

Evaluation of the initial concentration of sugars in the medium in jar fermenter culture (range of reagent glucose concentrations below 240 g/L)

### [Test Tube Culture (Pre-Culture)]

Test tube culture was performed under the same conditions as in Example 6 as a pre-culture.

### [Culture in 250 mL Jar Fermenter Culture (Main Culture)].

The jar fermenter and aeration conditions used were the same as in Example 6. The initial glucose concentration conditions for the medium were set to 60 g/L (standard), 100 g/L, 140 g/L, 180 g/L, 210 g/L, and 240 g/L. The medium composition with a reagent glucose concentration of 60 g/L (post-inoculation) in the modified CYM medium of Example 6 served as the standard condition. For the other glucose concentrations in the media, the concentrations of other medium components were increased in proportion to the increase in glucose concentration from the standard medium (60 g/L glucose concentration). Each of these media, 95 mL, was placed in 250 mL culture vessels. After mixing the test tube cultures, 5 mL of pre-culture was inoculated into each main culture vessel, and batch culture was started under the various glucose concentration conditions. The conditions for pH, DO, and agitation speed were the same as in Example 6. The main culture was continued for 144 to 240 hours.

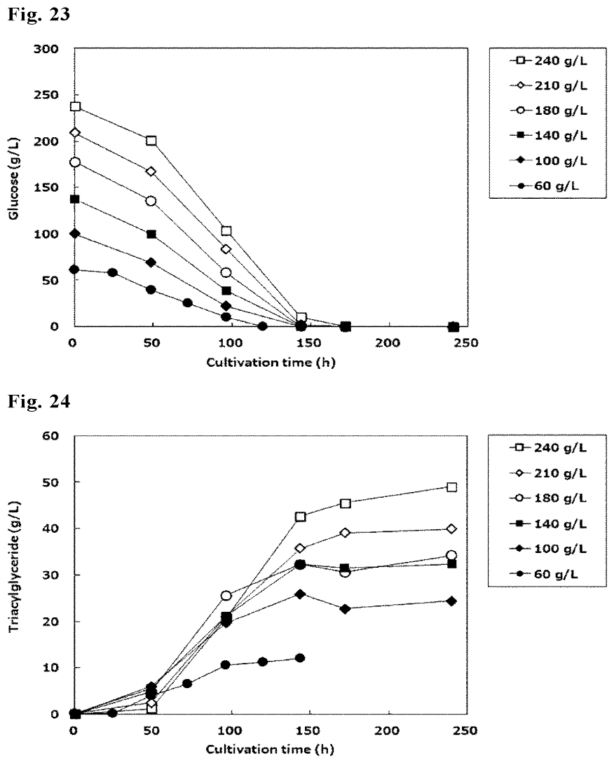

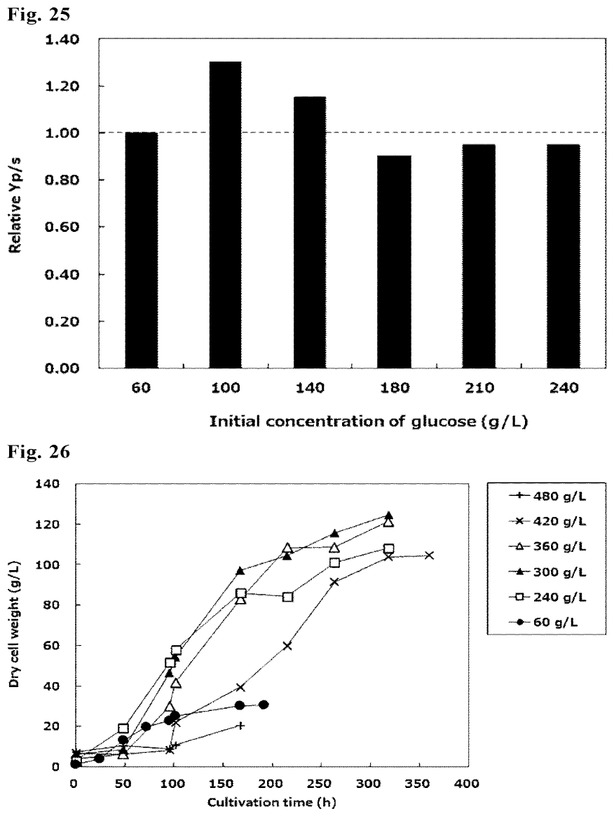

FIGS. 22, 23, and 24 show the changes over time in dry cell weight, glucose concentration, and triacylglyceride production of IS-300 strain under each glucose concentration condition, respectively. Cell growth and triacylglyceride production were observed in the glucose concentration range of 60 g/L to 240 g/L. FIG. 25 also shows the yield of triacylglyceride per sugar at each initial glucose concentration condition (relative to the 60 g/L glucose culture).

### Example 11

Evaluation of the initial concentration of sugars in the medium in jar fermenter culture (high concentration)

### [Test Tube Culture (Pre-Culture)]

Test tube culture was performed under the same conditions as in Example 6 as a pre-culture.

### [Culture in 250 mL Jar Fermenter Culture (Main Culture)].

The jar fermenter and aeration conditions used were the same as in Example 6.

The initial glucose concentration conditions for the medium were set to 60 g/L (standard), 240 g/L, 300 g/L, 360 g/L, 420 g/L, and 480 g/L. The medium composition with a reagent glucose concentration of 60 g/L (post-inoculation) in the modified CYM medium of Example 6 served as the standard condition. For the other glucose concentrations in the media, the concentrations of other medium components were increased in proportion to the increase in glucose concentration from the standard medium (60 g/L glucose concentration). Each of these media, 95 mL, was prepared in a 250 mL culture vessel. After mixing the test tube cultures, 5 mL of pre-culture was inoculated into each main culture vessel, and batch culture was started under the various glucose concentration conditions. The conditions for pH, DO, and agitation speed were the same as in Example 6. The main culture was continued for 192 to 360 hours.

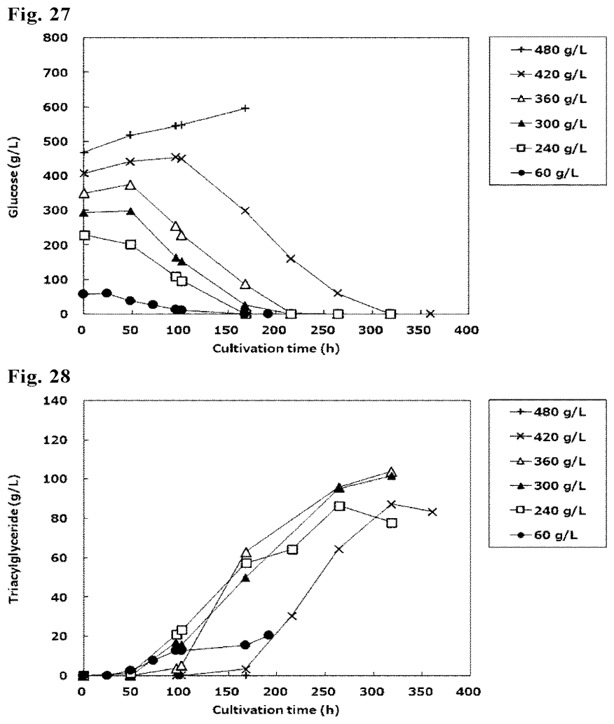

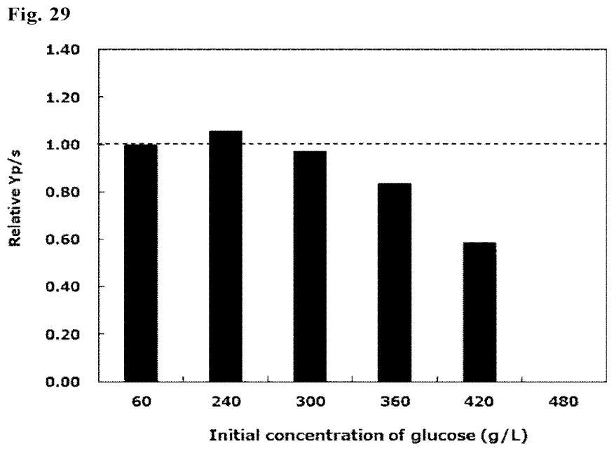

FIGS. 26, 27, and 28 show the changes over time in dry cell weight, glucose concentration, and triacylglyceride production of IS-300 strain under each initial glucose concentration condition, respectively. Cell growth and triacylglyceride production were observed in the glucose concentration range of 60 g/L to 420 g/L. No triacylglyceride production was observed at a glucose concentration of 480 g/L. FIG. 29 shows the yield of triacylglyceride per sugar at each glucose concentration (relative to the 60 g/L glucose culture).

1. A method for producing fats and oils, comprising:
culturing a yeast belonging to Leucosporidium golubevii in a medium containing sugar to produce fats and oils from the sugar; and
collecting the fats and oils.

2. The method of claim 1, wherein the medium containing sugar is a medium containing saccharified product of lignocellulosic biomass.

3. The method of claim 1, wherein the culture temperature is between 10° C. and 30° C.

4. The method of claim 1, wherein the culture temperature is 25° C. to 30° C.

5. The method of claim 1, wherein the sugar concentration is 50 g/L to 500 g/L.

6. The method of claim 1, wherein the sugar concentration is 200 g/L to 500 g/L.

7. The method of claim 1, wherein the yeast belonging to Leucosporidium golubevii is Leucosporidium golubevii IS-300 (NITE BP-03675) strain or a related strain thereof.

8. The method of claim 7, wherein the related strain is a strain having a nucleotide sequence including 5.8S rDNA region and ITS1 and ITS2 regions which is at least 95% identical to SEQ ID NO: 1, and
the related strain has fats and oils production ability at 28° C. which is equivalent to that at 24° C.

9. The method of claim 1, wherein the fats and oils are raw materials for biofuels, bionaphtha, food oil, lubricating oil, or surfactants.

10. Fats and oils produced by the method of claim 1.

11. Biofuel or bionaphtha made from fats and oils according to claim 10.

12. Leucosporidium golubevii IS-300 (NITE BP-03675) strain.
